(19) United States
(12) Patent Application Publication
(10) Pub. No.: US 2024/0008757 A1
(43) Pub. Date: Jan. 11, 2024

(54) HANDHELD LASER-BASED PERFUSION IMAGING APPARATUS AND METHOD OF USING SAID APPARATUS

(71) Applicant: UNIVERSITEIT TWENTE, Enschede (NL)

(72) Inventors: Wiendelt STEENBERGEN, Enschede (NL); Ata CHIZARI, Enschede (NL); Tom KNOP, Enschede (NL)

(21) Appl. No.: 18/272,993

(22) PCT Filed: Jan. 14, 2022
(86) PCT No.: PCT/NL2022/050010

§ 371 Date: Jul. 18, 2023

(30) Foreign Application Priority Data
Jan. 18, 2021 (NL) 2027331

(51) Int. Cl. A61B 5/026

(52) U.S. Cl. CPC A61B 5/0261

(57) ABSTRACT

Disclosed is a handheld laser-based perfusion imaging apparatus having a light source and an imaging device which are arranged in a fixed orientation to each other in the apparatus. The light source is configured for projecting a beam of coherent light onto a measurement field at a predetermined distance spaced apart from the apparatus. The imaging device is configured for recording speckle intensity maps of the measurement field and/or images of Doppler shifted light of the measurement field. The light source is configured to provide a substantially spherical wavefront or a substantial planar wavefront, at least at the measurement field. Preferably, the apparatus with the light source that provides a substantial planar wavefront, including a gimbal mount. Also disclosed is a method for measuring a perfusion in a tissue using the handheld laser-based perfusion imaging apparatus as described above.

### BACKGROUND

The invention relates to a handheld laser-based perfusion imaging apparatus and method of using said apparatus. Examples of laser-based perfusion imaging comprises Laser Speckle Contrast Imaging and Laser Doppler Perfusion Imaging.

Laser speckle contrast imaging (LSCI) is a well-known technique to study microcirculatory blood flow. The tissue of a subject is illuminated with coherent light and the backscattered light forms a so-called speckle pattern on the imaging sensor array. Due to the interaction of light with moving red blood cells within the tissue, the speckle patterns become time dependent. Different flow levels of red blood cells will cause different blurring levels of the time integrated speckle patterns. The parameter speckle contrast is used to evaluate the actual flow of the red blood cells.

As speckles are formed by the positive and negative interference of the coherent light on the subject, the speckle pattern is highly sensitive for any motions between the LSCI apparatus and the tissue measured. The sensitivity of speckle patterns to small movements necessitates that during the measurement, no other sources of movements should exist in order to form a reliable perfusion map. Any movements will cause motion artifacts in the measurements and will increase the error in the measurements of the perfusion assessment.

WO2020/064737 disclosed a handheld sensor head for LSCI measurements, wherein the sensor head comprises a movement sensor configured to output a movement signal representing a movement taking place during the period of time of acquiring the image data, and a processing device configured to receive the image data and the movement signal and to output movement corrected image data.

Laser Doppler perfusion imaging (LDPI) is also a diagnostic method to measure blood flow in tissue. The tissue of a subject is illuminated with coherent light and the backscattered light forms a so-called speckle pattern on the imaging sensor array, and again, due to the interaction of light with moving red blood cells within the tissue, the speckle patterns become time dependent. For LDPI measurements, the imaging sensor array is configured to collect a series of speckle pattern images at a high frame rate such that each individual speckle pattern image of said series of speckle pattern images is substantially not blurred. The series of speckle pattern images is converted in an intensity signal as a function of time for each pixel in the speckle pattern image. By analyzing the frequency content of the fluctuations in the intensity signal, in particular an average frequency of said fluctuations, and the variance of said fluctuations in the signal, the perfusion in said tissue at the position of a pixel can be characterized. Again, any relative movements between the studied tissue and the measuring device, will cause motion artifacts in the measurements and will increase the error in the measurements of the perfusion assessment. This can be either by tissue motion or by movements of the imaging apparatus.

### SUMMARY OF THE INVENTION

A problem of the use of a movement sensor and processing the data to correct for movement taking place during the period of time of acquiring the image data is, that it is sometimes very difficult to distinguish between a blurring of the speckle pattern due to the flow of the red blood cells and a blurring of the speckle pattern due to motion artifacts.

It is an object of the present invention to provide a measurements arrangement for a laser-based perfusion imaging apparatus, which is much more forgiving to small movements that may occur during the measurement, and therefore is less sensitive to small motion artifacts.

According to a first aspect, the present invention pertains to a handheld laser-based perfusion imaging apparatus comprising a light source and an imaging device which are arranged in a fixed orientation to each other in said apparatus, wherein
the light source is configured for projecting a beam of coherent light onto a measurement field at a predetermined distance spaced apart from the apparatus,
the imaging device is configured for recording intensity maps of the measurement field,
wherein the light source comprises one or more optical components which are configured to provide that the beam of coherent light comprises a spherical wavefront, wherein the wavefront is convex towards the measurement field.

The inventors have found that at least movement artefacts due to the rotation of the laser-based perfusion imaging apparatus can be reduced. Accordingly, by configuring the laser-based perfusion imaging apparatus so that the coherent light source provides a spherical wavefront at the measurement field, such that the wavefront is bulging towards the measurement field, the apparatus of the present invention is much more forgiving to rotational movements of the handheld apparatus which may occur during the measurement, and therefore the apparatus of the present invention is less sensitive to small motion artifacts due to the handheld operation of the apparatus.

For example, the inventors found that the speckle contrast decreases with increasing rotational speed. However this decrease is much less for an LSCI apparatus which configured to emit coherent light with a spherical wavefront, when compared to an LSCI apparatus which is configured to emit coherent light with e.g. a planar wavefront or a scrambled wavefront (by using an optical diffuser for example).

In an embodiment, the one or more optical components are configured to position a center of the spherical wavefront at or near a pivot point of said handheld laser-based perfusion imaging apparatus and/or a pivot point of a user of said handheld laser-based perfusion imaging apparatus. Within the context of this description, the center of the spherical wavefront is the actual or virtual position with substantially the same distance to all positions on the spherical wavefront. The center can also be seen as an origin from where the electromagnetic waves of the coherent light source are or seem to be emanating. By arranging the center of the spherical wavefront at or near a pivot point of said handheld laser-based perfusion imaging apparatus, for example at or near a grip or handle for holding the apparatus, the sensitivity of the apparatus of the present invention to rotational movements of the handheld apparatus can be further reduced, in particular for rotational movements around said center which may occur during the measurement. In addition or alternatively, by arranging the center of the spherical wavefront at or near a pivot point of a user of said handheld laser-based perfusion imaging apparatus, for example at or near a wrist, elbow or shoulder joint of a user holding the grip or handle, the sensitivity of the apparatus of the present invention to rotational movements of the handheld apparatus can be further reduced, in particular for rotational movements around said pivot point of the user which may occur during the measurement. Preferably, the position of the center of the spherical wavefront with respect to the handle or grip of the handheld laser-based perfusion imaging apparatus, is based on anthropometric data, for example from an anthropometric database, preferably based on the average anthropometric data of a group of intended users, or based on anthropometric data as presented in a National Standard (see for example NTA 8500:2007 nl).

In an embodiment, the one or more optical components comprises a single mode optical fibre, which is configured for emitting a diverging light beam. Single mode optical fibres usually have a core with a very small diameter, for example in a range of 8-10 micrometres, and accordingly the end of a single mode optical fibre closely approximates a point source, emitting a diverging light beam within a cone of light. Within said cone of light, the wavefront is substantially spherical, wherein said wavefront is substantially convex towards the measurement field, thus the wavefront is substantially bulging away from the end of the single mode optical fiber. Preferably, a coherent light emitting end of said single mode optical fiber or an actual or virtual image thereof is arranged at or near a pivot point of said handheld laser-based perfusion imaging apparatus and/or a pivot point of a user of said handheld laser-based perfusion imaging apparatus.

A further advantage of the use of a single mode optical fiber is, that the optical fiber allows to arrange the actual source of light, in particular a laser, spaced apart from the light source, in particular the end of the single mode optical fibre that is configured for projecting a beam of coherent light onto a measurement field at a predetermined distance spaced apart from the apparatus.

In an embodiment, the one or more optical components comprises a focusing lens and a pinhole aperture arranged at a position where the focusing lens focusses the beam of coherent light. In such an arrangement, the light from the light source, in particular a laser, is focused to a small spot size. In the focus position, a pinhole is arranged, wherein the pinhole has a diameter between 1 and 1,5 times the beam spot size in said focus. Typically, the pinhole has a diameter in a range of 4 to 12 micrometres. The pinhole aperture closely approximates a point source, which produces light that approximates a spherical wavefront. It is noted that a smaller aperture implements a closer approximation of a point source, which in turn produces a more nearly spherical wavefront, but the transmitted power is greatly reduced. It is noted that assemblies comprising a focusing lens and a pinhole aperture, are known in the art. Preferably, the pinhole or an actual or virtual image thereof is arranged at or near a pivot point of said handheld laser-based perfusion imaging apparatus and/or a pivot point of a user of said handheld laser-based perfusion imaging apparatus.

In an embodiment, the light source is configured to provide a substantially collimated light beam and wherein the one or more optical components comprises a lens, preferably a negative lens, for converting the substantially collimated light beam in a diverging light beam with a spherical wavefront. The lens converts the collimated light beam into a diverging light beam. In case of a negative lens, the diverging light beam seems to be emitted from a virtual focus point, which is arranged between the light source and the negative lens. In case of a positive lens, the light from the light source is first focused in a focus point of the positive lens, wherein focus point is arranged at a side of the positive lens facing away from the light source, and after the focus point the light beam is a diverging light beam. The virtual focus point of the negative lens or focus point of the positive lens approximates a point source, which produces light that approximates a spherical wavefront. Preferably, the virtual focus point of the negative lens or focus point of the positive lens or an actual or virtual image thereof is arranged at or near a pivot point of said handheld laser-based perfusion imaging apparatus and/or a pivot point of a user of said handheld laser-based perfusion imaging apparatus.

In an embodiment, the light source comprises one or more further optical components which are configured for converting the spherical wavefront into a planar wavefront. The inventors have found that at least movement artefacts due to a translation of the handheld laser-based perfusion imaging apparatus can be reduced. In particular a translation substantially parallel to the planar wavefront. Accordingly, by configuring the handheld laser-based perfusion imaging apparatus so that the coherent light source provides a planar wavefront at the measurement field, preferably such that the wavefront is substantially parallel to the measurement field, the apparatus of the present invention is much more forgiving to translational movements of the handheld apparatus which may occur during the measurement, and therefore the apparatus of the present invention is less sensitive to small motion artifacts due to the handheld operation of the apparatus.

For example, in an LSCI apparatus, the inventors found the speckle contrast decreases with increasing translational speed. However this decrease is much less for an LSCI apparatus which configured to emit coherent light with a planar wavefront, when compared to an LSCI apparatus which is configured to emit coherent light with a spherical wavefront or a scrambled wavefront.

It is noted that the above embodiment, wherein the light source is configured to provide a substantially planar wavefront at the measurement field, is less forgiving to rotational movements. However, in order to decrease the sensitivity to rotational movements of a handheld laser-based perfusion imaging apparatus with a light source configured to provide a substantially planar wavefront, in an embodiment, the handheld laser-based perfusion imaging apparatus comprises a gimbal mount. The gimbal mount is preferably arranged in between the assembly of the light source and the imaging device on the one hand, and a handle or grip for manually holding the handheld laser-based perfusion imaging apparatus. The gimbal mount is configured to absorb at least a part of a rotational movement of the handle or grip by the operator, which may occur during the measurement. Accordingly, due to the additional gimbal mount the apparatus according to this embodiment is more forgiving to rotational movements of the handheld apparatus due to the gimbal mount and more forgiving to translational movements of the handheld apparatus due to the planar wavefront.

In an embodiment, the one or more further optical components comprises collimating lenses and/or a beam expander. Accordingly, these further optical components allow to adjust the diameter of the beam of coherent light so that it substantially completely illuminates the measurement field with a desired size.

According to a second aspect, the present invention pertains to a handheld Laser Speckle Contrast Imaging (LSCI) apparatus or Laser Doppler Perfusion Imaging (LDPI) apparatus comprising a light source and an imaging device which are arranged in a fixed orientation to each other in said apparatus, wherein
the light source is configured for projecting a beam of coherent light onto a measurement field at a predetermined distance spaced apart from the apparatus,
the imaging device is configured for recording the speckle intensity maps of the measurement field,
wherein the light source is configured to provide a substantial planar wavefront, at least at the measurement field.

In the LSCI or LDPI apparatus according to the second aspect of the invention, the light source is already configured to provide a substantial planar wavefront. Thus a conversion of a spherical wavefront into a planar wavefront, as in the apparatus according to the first aspect of the invention, is not necessary.

As already noted above, by configuring the apparatus so that the coherent light source provides a planar wavefront at the measurement field, preferably such that the wavefront is substantially parallel to the measurement field, the apparatus of the present invention is much more forgiving to translational movements of the handheld apparatus which may occur during the measurement, and therefore the apparatus of the present invention is less sensitive to small motion artifacts due to the handheld operation of the apparatus.

It is noted that, in the LSCI apparatus, the imaging device is configured to capture time integrated speckle images, and the blurring of said time integrated speckle images is used to characterize the perfusion. Whereas, in the LDPI apparatus, the imaging device is configured to capture a series of speckle images at a high frame rate in order to obtain a series of substantially non-blurred speckle images, and the frequency content of the intensity fluctuations in each pixel during the timeframe that the series of speckle images is recorded, is used to characterize the perfusion at the position of the tissue that corresponds to said pixel. Accordingly, the LSCI apparatus and the LDPI apparatus is very much alike, except that the LDPI apparatus requires a high speed imaging device and the LSCI apparatus uses a low speed time integrating imaging device, and both use different analysis methods to characterize the perfusion.

In an embodiment of the apparatus according to the second aspect of the invention, the apparatus comprises a gimbal mount. Accordingly, due to the additional gimbal mount the apparatus according to this embodiment is more forgiving to rotational movements of the handheld apparatus due to the gimbal mount and more forgiving to translational movements of the handheld apparatus due to the planar wavefront.

In an embodiment, the light source comprises one or more further optical components, wherein said one or more further optical components comprises a beam expander. Accordingly, these further optical components allow to adjust the diameter of the beam of coherent light so that it substantially completely illuminates the measurement field of a desired size.

In an embodiment, the apparatus comprises one or more targeting light sources which are configured to project one or more targeting light beams onto the measurement field, preferably wherein the one or more targeting light sources comprises two cross-line laser modules which are configured to illuminate the boundaries of the measurement field. On the one hand, the one or more targeting light sources allows to accurately position the measurement field at the desired spot where the tissue perfusion level needs to be measured. However, since the targeting light beams are visible at or near the measurement field by the operator, the targeting light beams also provide feedback to the operator of any movements of the handheld apparatus which may occur during the measurement. This feedback may be used by the operator to keep the handheld LSCI apparatus more steady and thus to substantially reduce the occurrence of motion artifacts.

According to a third aspect, the present invention pertains to a method for measuring a perfusion in a tissue using a handheld Laser Speckle Contrast Imaging (LSCI) apparatus, wherein the method comprises the steps of:
projecting a beam of coherent light onto a measurement field at a predetermined distance spaced apart from the apparatus,
recording time integrated speckle intensity maps of the measurement field,
wherein the apparatus comprises a light source comprising one or more optical components for configuring the beam of coherent light to comprise a spherical wavefront at least at the measurement field, wherein the wavefront is convex towards the measurement field.

Preferably, the blurring of said time integrated speckle images is used to characterize the perfusion.

According to a fourth aspect, the present invention pertains to a method for measuring a perfusion in a tissue using a handheld Laser Doppler Perfusion Imaging (LDPI) apparatus, wherein the method comprises the steps of:
projecting a beam of coherent light onto a measurement field at a predetermined distance spaced apart from the apparatus,
recording a series of speckle images at a high frame rate and/or short exposure times in order to obtain a series of substantially non-blurred speckle images,
wherein the apparatus comprises a light source comprising one or more optical components for configuring the beam of coherent light to comprise a spherical wavefront at least at the measurement field, wherein the wavefront is convex towards the measurement field.

Preferably, the frequency content of the intensity fluctuations in each pixel during the timeframe that the series of speckle images is recorded, is used to characterize the perfusion at the position of the tissue that corresponds to said pixel.

In an embodiment of the method according to the third or fourth aspect of the invention, the light source comprises one or more further optical components which are configured for converting the spherical wavefront into a planar wavefront. It is noted that the above methods provide the same advantages and/or solve the same problems as described above regarding the apparatus according to the first aspect.

According to a fifth aspect, the present invention pertains to a method for measuring a perfusion in a tissue using a handheld Laser Speckle Contrast Imaging (LSCI) apparatus, wherein the method comprises the steps of:
projecting a beam of coherent light onto a measurement field at a predetermined distance spaced apart from the apparatus,
recording time integrated speckle intensity maps of the measurement field,
wherein the apparatus comprises a light source, wherein the light source is configured to provide a substantial planar wavefront, at least at the measurement field.

Preferably, the blurring of said time integrated speckle images is used to characterize the perfusion.

According to a sixth aspect, the present invention pertains to a method for measuring a perfusion in a tissue using a handheld Laser Doppler Perfusion Imaging (LDPI) apparatus, wherein the method comprises the steps of:
projecting a beam of coherent light onto a measurement field at a predetermined distance spaced apart from the apparatus,
recording a series of speckle images at a high frame rate in order to obtain a series of substantially non-blurred speckle images, wherein the apparatus comprises a light source, wherein the light source is configured to provide a substantial planar wavefront, at least at the measurement field.

Preferably, the frequency content of the intensity fluctuations in each pixel during the timeframe that the series of speckle images is recorded, is used to characterize the perfusion at the position of the tissue that corresponds to said pixel.

In an embodiment of the method according to the fifth or sixth aspect of the invention, the apparatus comprises a gimbal mount. It is noted that the above methods provide the same advantages and/or solve the same problems as described above regarding the apparatus according to the second aspect

The various aspects and features described and shown in the specification can be applied, individually, wherever possible. These individual aspects, in particular the aspects and features described in the attached dependent claims, can be made subject of divisional Patent Applications.

### BRIEF DESCRIPTION OF THE DRAWINGS

The invention will be elucidated on the basis of an exemplary embodiment shown in the attached drawings, in which:

FIG. 1 shows a schematic example of an apparatus for laser-based perfusion imaging according to the present invention,

FIG. 2 schematically shows a first exemplary embodiment of the apparatus of FIG. 1 with a light source which emits light with a spherical wavefront,

FIG. 3 schematically shows an alternative light source for use in the exemplary embodiment of FIG. 2,

FIG. 4 schematically shows a second exemplary embodiment of the apparatus of FIG. 1 with a light source which emits light with a planar wavefront,

FIG. 5 schematically shows an alternative light source for use in the exemplary embodiment of FIG. 4,

FIGS. 6 and 7 schematically show a third exemplary embodiment of a handheld laser-based perfusion imaging apparatus according to the invention, and

FIGS. 8A and 8B schematically show a fourth exemplary embodiment of a handheld laser-based perfusion imaging apparatus according to the invention.

### DETAILED DESCRIPTION OF THE INVENTION

Laser speckle contrast imaging (LSCI) is a well-known technique to study microcirculatory blood flow. The tissue is illuminated with coherent light and the backscattered light forms a so-called speckle pattern on the imaging sensor array. Due to the interaction of light with moving red blood cells (RBCs) within the tissue, the speckle patterns become time dependent. Different flow levels of RBCs will cause different blurring levels of the time integrated speckle patterns. The speckle contrast is used as a parameter to evaluate the actual flow. However, relative movements between the LSCI apparatus and the studied tissue will also induce a blurring of the speckle patterns.

Laser Doppler perfusion imaging (LDPI) is a diagnostic method to measure blood flow in tissue. The tissue of a subject is illuminated with coherent light and the backscattered light forms a so-called speckle pattern on the imaging sensor array. Due to the interaction of light with moving red blood cells (RBCs) within the tissue, the speckle patterns become time dependent. For LDPI measurements, the imaging sensor array is configured to collect a series of speckle pattern images at a high frame rate or short exposure times such that each individual speckle pattern image of said series of speckle pattern images is substantially not blurred. The series of speckle pattern images is converted in an intensity signal as a function of time for each pixel in the speckle pattern image. By analyzing the frequency content of the fluctuations in the intensity signal, in particular an average frequency of said fluctuations, and the variance of said fluctuations in the signal, the perfusion in said tissue at the position of a pixel can be characterized. Different flow levels of RBCs will cause different intensity fluctuations in the speckle images. However, relative movements between the LDPI apparatus and the studied tissue will also induce intensity fluctuations in the speckle images.

The sensitivity of laser-based perfusion imaging, such as LSCI or LDPI, to small movements necessitates that during the measurement, no other source of movements should exist in order to form a reliable perfusion map. From one side, patient movements originated by breathing, heartbeat and organ tremor is a source of movement artefacts. From another side, when using handheld, operator-generated movements of the laser-based perfusion imaging system caused by the operator are another source of movement artefacts. Therefore, practical realization of such experimental environment remains a challenge.

The present invention provides a compact and handheld laser-based perfusion imaging apparatus in order to perform measurements on various patient body areas without inconvenience for patients or investigators, and which apparatus is less sensitive to movement artefacts.

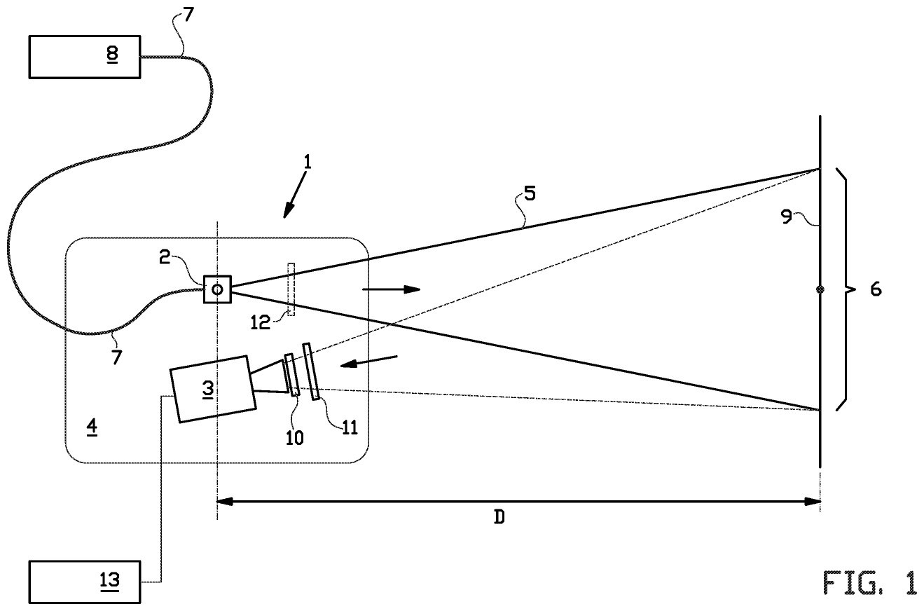

An example of an apparatus according to the present invention is shown in FIG. 1. The handheld laser-based perfusion imaging apparatus 1 comprises a light source 2 and an imaging device 3 which are arranged in a fixed orientation to each other in said apparatus 1. In this example the light source 2 and the imaging device 3 are mounted on a platform 4. The light source 2 is configured for projecting a light beam 5 of coherent light onto a measurement field 6, preferably at a distance D spaced apart from the apparatus 1. The imaging device 3 is configured for recording the speckle intensity maps of the measurement field 6.

The light source 2 in this example is provided by a tip at a distal end of a single mode optical fiber 7. The proximal end of the single mode optical fiber 7 is connected to a source of coherent light. In this particular example, a continuous wave single longitudinal mode laser 8 and a coherence length of longer than an optical path length difference between the shortest and longest photon trajectories, in particular the optical path length difference between the shortest and longest photon trajectories through the tissue, is used. The light of said laser 8 is coupled into the single mode fiber 7.

In case the laser-based perfusion imaging apparatus 1 comprises a LSCI or an LDPI apparatus, the imaging device 3 may comprise a monochrome camera, which is mounted on the platform 4 to record the speckle intensity maps.

A bandpass interference filter 10, which is configured to allow light with a wavelength of the light from the laser 8 to pass, is mounted in front of the camera objective in order to reduce the background light.

In addition, in case the output of the laser 8 is linearly polarized, the detection of specular reflection can at least partially be avoided by arranging a linear polarizer 11 with the appropriate orientation in front of the imaging device 3. In order to maintain the polarization of the laser light from the laser 8, a polarization maintaining optical fiber 7 may be used. In case the polarization is partly lost in the single mode optical fiber 7, the polarization may be at least partially be restored by optionally arranging a polarizer 12 with the appropriate orientation in the light beam 5.

It is noted that the single mode optical fiber 8 prevents speckle change due to the movements of handheld LSCI or LDPI apparatus 1.

In case the laser-based perfusion imaging apparatus 1 comprises a LSCI apparatus, the imaging device 3 is configured to acquire time integrated speckle intensity maps.

In case the laser-based perfusion imaging apparatus 1 comprises a LDPI apparatus, the imaging device 3 may comprise a camera with a high frame rate and/or a short exposure time, which is mounted on the platform 4 to record the series of non-blurred speckle intensity maps.

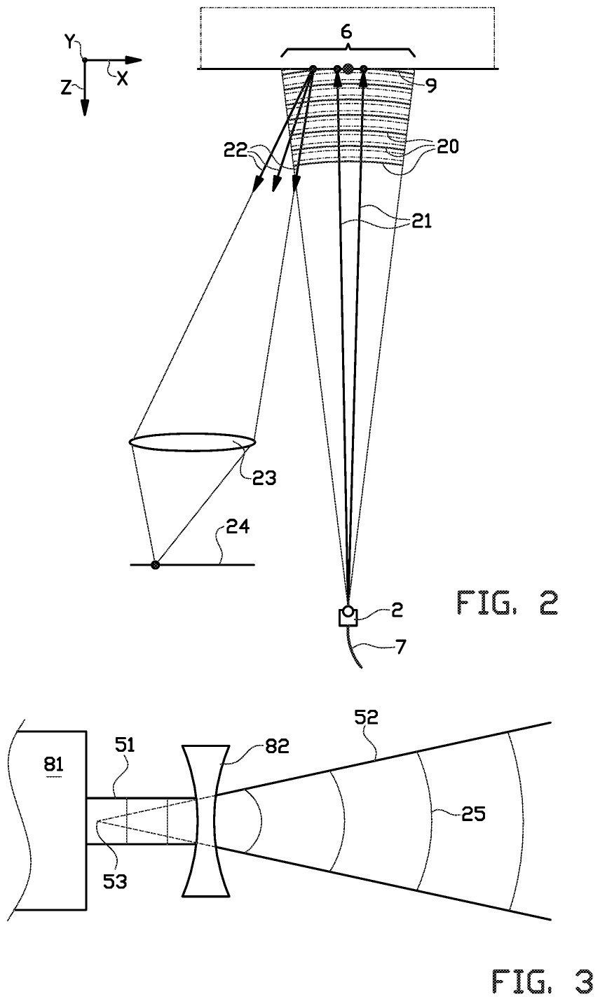

FIG. 2 schematically shows the illumination and imaging system of the example of FIG. 1 in a first exemplary embodiment according to the invention, in which the surface 9 of the measuring field 6 is illuminated with a light beam 5 having a spherical wavefront 20. The spherical wavefront 20 is convex towards the measurement field 6. The center of the spherical wavefronts 20 is arranged at the light source 2, in particular at the tip of the optical fiber 7. Also schematically shown in FIG. 2 are incoming wave-vectors 21, which are perpendicular to the wavefront 20, which are directed toward the measuring field 6. The outgoing wave-vectors 22 are collected by the imaging lens 23 (camera objective) and imaged on the sensor 24 of the camera 3.

The data from the sensor 24 of the camera 3 is transferred to a computing device 13, as schematically shown in FIG. 1.

In case the perfusion device 1 is a LSCI apparatus, the computing device 13 is configured to calculate the speckle contrast for a time-integrated image as captured by the camera 3, and to determine a measure for the perfusion based on the calculated speckle contrast.

In case the perfusion device 1 is a LDPI apparatus, the computing device 13 is configured to For LDPI measurements, the imaging sensor array is configured to convert the collected series of non-blurred speckle pattern images at a high frame rate or short exposure times in an intensity signal as a function of time for each pixel in the speckle pattern image, and analyze the frequency content of the fluctuations in the intensity signal, in particular an average frequency of said fluctuations, and the variance of said fluctuations in the signal, to determine a measure for the perfusion in said tissue at the position of a pixel.

The inventors have found that at least movement artefacts due to the rotation of the laser-based perfusion imaging apparatus 1, in particular a rotation around the x-axis and/or y-axis, can be reduced. Accordingly, by configuring the laser-based perfusion imaging apparatus 1 so that the coherent light source 2 provides a spherical wavefront 20 at the measurement field 6, such that the wavefront 20 is bulging towards the measurement field 6, the apparatus of the present invention is much more forgiving to rotational movements of the handheld apparatus which may occur during the measurement. Therefore, the apparatus 1 of the present invention is less sensitive to small motion artifacts due to the handheld operation of the apparatus.

It is noted that, in the example of FIG. 2, the center of the spherical wavefront 20 is arranged at or near the light source 2, in particular at the tip of the optical fiber 7. Accordingly, it is preferred to arrange the light source 2 at or near a pivot point of said handheld laser-based perfusion imaging apparatus 1 and/or at or near a handle or grip of the handheld laser-based perfusion imaging apparatus 1. Furthermore, the distance D is preferably the distance between the light source 2 and the measurement field 6.

FIG. 3 schematically shows an example of an alternative light source for use in the first exemplary embodiment. In particular, FIG. 3 shows a laser 81 which in an alternative embodiment is arranged on the platform 4, instead of the light source 2. The laser 81 is configured to provide a substantially collimated light beam 51. In front of the laser 81, a negative lens 82 is arranged for converting the substantially collimated light beam 51 in a diverging light beam 52 with a spherical wavefront 25. The diverging light beam 52 seems to be emitted from a virtual focus point 53.

It is noted that instead of a negative lens 82, also a positive lens can be used. However, in this case, the light from the laser 81 is first focused in a focus point of the positive lens, which focus point is arranged at a side of the positive lens facing away from the laser 81, and after the focus point the light beam is a diverging light beam.

It is further noted that, in the example of FIG. 3, the virtual center of the spherical wavefront 25 is arranged at the virtual focus point 53. Accordingly, by selecting the appropriate lens 82, the position of the center of the spherical wavefront 25 can be arranged at or near a pivot point of said handheld laser-based perfusion imaging apparatus 1 and/or at or near a handle or grip of the handheld laser-based perfusion imaging apparatus 1. The center 53 of the spherical wavefront 25 can be arranged at or near a pivot point of said handheld laser-based perfusion imaging apparatus 1, for example at or near a grip or handle for holding the apparatus 1. Alternatively, the center 53 of the spherical wavefront 25 can be arranged outside the handheld laser-based perfusion imaging apparatus 1, in particular for arranging the center 53 of the spherical wavefront 25 at or near a pivot point of a user of said handheld laser-based perfusion imaging apparatus 1, for example at or near a wrist, elbow or shoulder joint of a user.

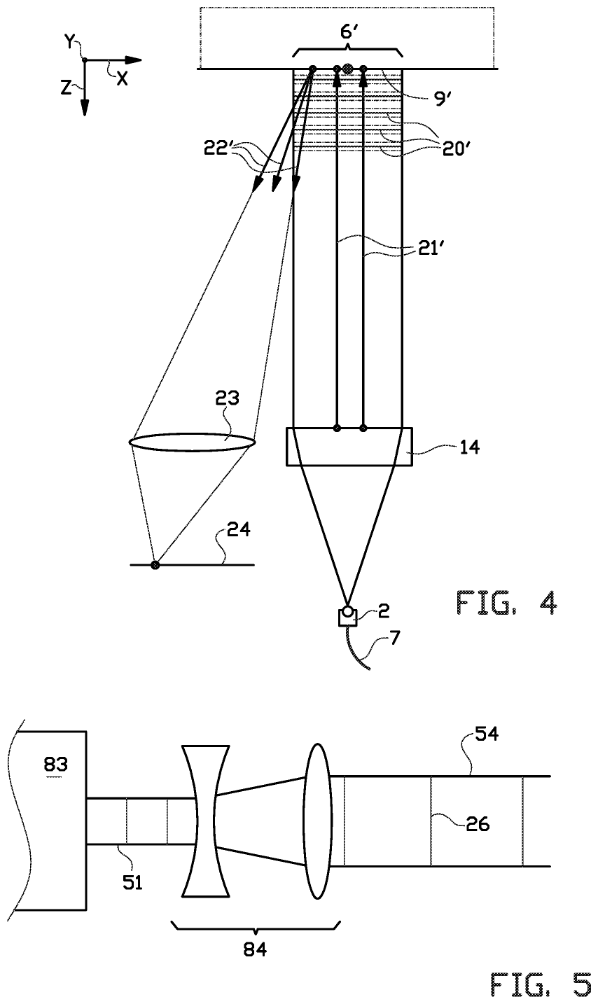

FIG. 4 schematically shows the illumination and imaging system of the example of FIG. 1 in a second exemplary embodiment according to the invention, in which the surface 9′ of the measuring field 6′ is illuminated with a light beam 5′ having a substantially planar wavefront 20′.

The light source 2 in this example is again provided by a tip at a distal end of a single mode optical fiber 7. The tip of the optical fiber 7 emits a diverging beam, which is converted into a parallel beam 5′ by means of a collimator lens 14. In the area between the collimator lens 14 and the measuring field 6′, the wavefront 20′ of the light beam 5′ is substantially planar.

Also schematically shown in FIG. 4 are incoming wave-vectors 21′, which are perpendicular to the wavefront 20′, and which are directed toward the measuring field 6′. The outgoing wave-vectors 22′ are collected by the imaging lens 23 (camera objective) and imaged on the sensor 24 of the camera 3.

The data from the sensor 24 of the camera 3 are transferred to a computing device 13, as schematically shown in FIG. 1.

The inventors have found that at least movement artefacts due to a translation of the handheld laser-based perfusion imaging apparatus 1, in particular a translation substantially parallel to the xy-plane, can be reduced. Accordingly, by configuring the handheld laser-based perfusion imaging apparatus 1 so that the coherent light source 2 provides a planar wavefront 20′ at the measurement field 6′, the apparatus of the present invention is much more forgiving to translational movements of the handheld apparatus which may occur during the measurement. Therefore, the apparatus 1 of the present invention is less sensitive to small motion artifacts due to the handheld operation of the apparatus.

FIG. 5 schematically shows an example of an alternative light source for use in the second exemplary embodiment. In particular, FIG. 5 shows a laser 83 which in an alternative embodiment is arranged on the platform 4, instead of the light source 2. The laser 83 is configured to provide a substantially collimated light beam 51 with a substantially planar wavefront. In front of the laser 83, a beam expander 84 is arranged for converting the substantially collimated light beam 51 in a substantially collimated light beam 54 with a larger diameter in order to suitably illuminate the measurement field. The collimated light beam 54 is configured to project a coherent light beam with a planar wavefront 26 onto the measurement field 6′. Accordingly, the beam expander 84 allows to adjust the diameter of the measurement field 6′.

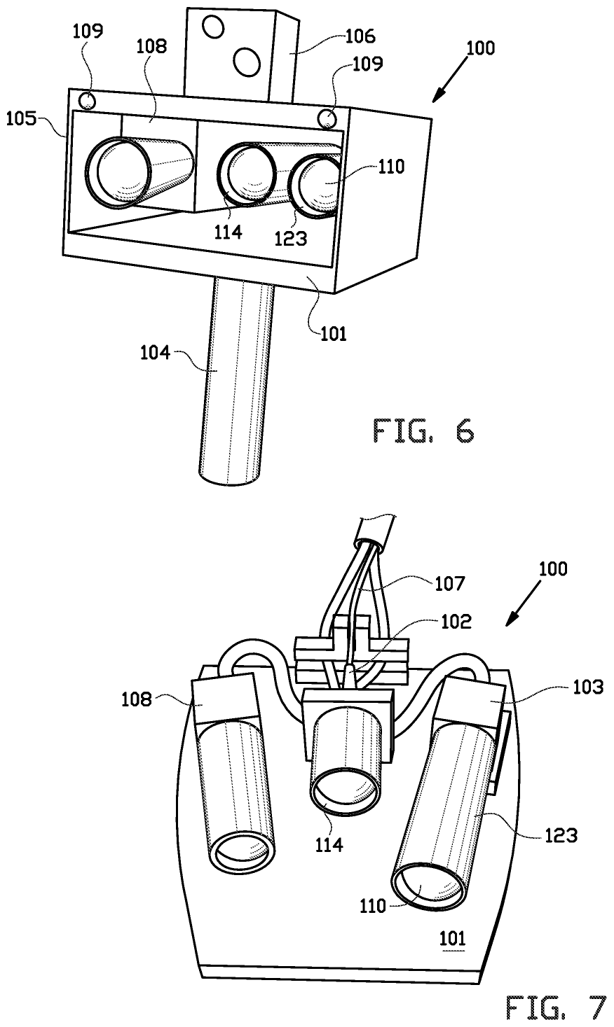

FIGS. 6 and 7 schematically show a third exemplary embodiment of a handheld laser-based perfusion imaging apparatus 100 according to the invention. In FIG. 7 the casing 105 is removed to shown the internal of the handheld laser-based perfusion imaging apparatus 100 more clearly.

For perfusion imaging, the laser-based perfusion imaging apparatus 100 comprises a coherent and continuous wave single longitudinal mode laser (not shown). The laser beam is coupled into a single mode optical fiber 107. The distal end of the optical fiber 107 is attached to the handheld probe 101, and connected to a projection lens 114. The projection lens 114 is arranged in front of the fiber tip, and is configured to illuminate a measurement field of a desired size at a desired distance from the apparatus 100 by the laser beam with a spherical wavefront.

The distance from the light source 102 and the sensors of the camera 103 to the tissue surface may been set to a convenience distance for performing the perfusion measurements, for example 40 cm, although it may vary slightly during handheld operations. The measured beam width, i.e. the radial distance at which the intensity decreases by a factor of 1/e² of its maximum at the center of illumination, for this system is configured to a desired size, for example approximately 8 cm. Due to the spherical wavefront, handheld laser-based perfusion imaging apparatus 100 is less sensitive to the movement artefacts due to the rotation of the probe 101.

Preferably, the intensity of the outputted laser beam is set based on calculation for maximum permissible exposure for the wavelength of the laser, for example using an eye pupil diameter of 7 mm and blink reflex of 0.2 seconds, so that the handheld laser-based perfusion imaging apparatus 100 is eye safe after the light exits the casing 105 of the handheld probe 101, which in this example is about 10 cm away from the fiber tip. Thus, the handheld laser-based perfusion imaging apparatus 100 can be operated without wearing laser safety goggles.

To allow or block laser illumination on the tissue, a motorized shutter (not shown) may be arranged in front of the lens 114 or in front of the light source 102.

In this example, as shown in FIG. 6, a targeting laser system 106, comprising two cross-line laser modules, is provided to illuminate the boundaries of the measurement field. This assists the operator in targeting the regions of interest. The incorporation of an aiming beam in the handheld system 100 will help investigators to keep the system more stable during handheld measurements, and thus to further reduce motion artefacts.

To record the speckle intensity patterns, a camera 103 is provided with a camera objective 123. The camera objective 123 is provided with a bandpass interference filter 110, wherein the bandpass interference filter 110 is configured to pass light with the same wavelength as the wavelength of the laser beam, in order to filter out most of the light from the surrounding lighting.

In addition, the probe 101 in the example shown in FIG. 6 is provided with a color camera 108 for making RGB images of at least the measurement field. Two high power light emitting diodes 109 (LEDs) are provided to illuminate at least the measurement field with a substantial white-light illumination to assist during the making of the RGB images.

As schematically indicated in FIG. 6, at the bottom side of the probe 101, a handle or grip 104 is provided.

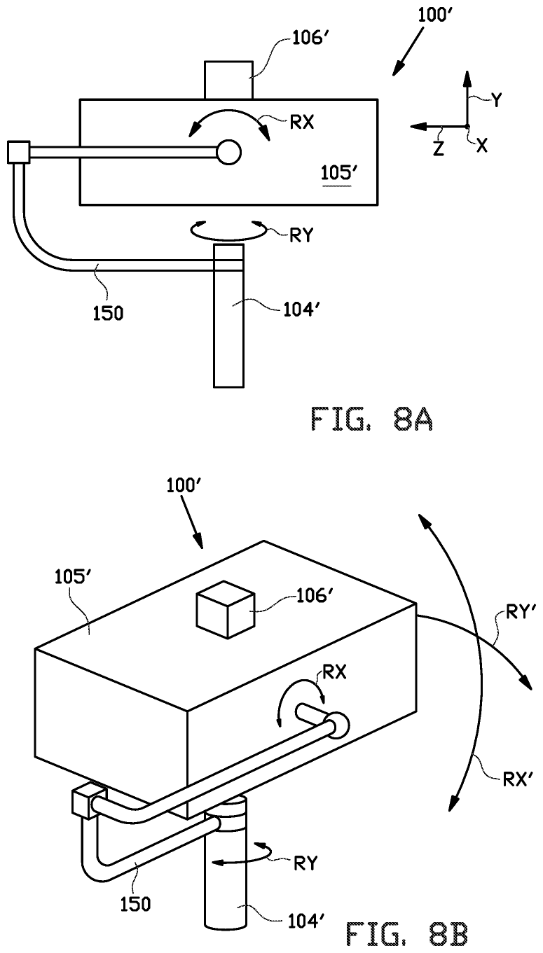

FIGS. 8A and 8B schematically show a fourth exemplary embodiment of a handheld laser-based perfusion imaging apparatus 100′ according to the invention. The laser-based perfusion imaging apparatus 100′ comprises almost the same arrangement as the laser-based perfusion imaging apparatus 100 of the third exemplary embodiment, except that the optics inside the apparatus 100′ is configured to provide a laser beam with a planar wavefront (for example using the arrangement as described above with reference to FIG. 4 or 5).

As already discussed above, the apparatus 100′ which is configured to provide a substantially planar wavefront at the measurement field, is less forgiving to rotational movements. In order to decrease the sensitivity to rotational movements of a laser-based perfusion imaging apparatus 100′ with a light source configured to provide a substantially planar wavefront, the laser-based perfusion imaging apparatus 100′ according to this fourth exemplary embodiment comprises a gimbal mount 150. The gimbal mount 105 is arranged in between the casing 105′ of the apparatus 100′ and a handle or grip 104′ for manually holding the laser-based perfusion imaging apparatus 100′. The gimbal mount 105 is configured to absorb at least a part of a rotational movement of the handle or grip 104′ by the operator, which may occur during the measurement. In particular, the gimbal mount 105 is rotatable around two orthogonal axes in a plane parallel to the measurement field, i.e. around the x-axis Rx and around the y-axis Ry as schematically shown in FIGS. 8A and 8B. Accordingly, due to the additional gimbal mount 150 the handheld laser-based perfusion imaging apparatus 100′ according to this fourth exemplary embodiment is more forgiving to rotational movements Rx′, Ry′ of the handheld apparatus 100′ and more forgiving to translational movements of the handheld apparatus 100′ due to the planar wavefront.

It is to be understood that the above description is included to illustrate the operation of the preferred embodiments and is not meant to limit the scope of the invention. From the above discussion, many variations will be apparent to one skilled in the art that would yet be encompassed by the scope of the present invention.

In summary, the invention relates to a handheld laser-based perfusion imaging apparatus comprising a light source and an imaging device which are arranged in a fixed orientation to each other in said apparatus. The light source is configured for projecting a beam of coherent light onto a measurement field at a predetermined distance spaced apart from the apparatus. The imaging device is configured for recording speckle intensity maps of the measurement field and/or images of Doppler shifted light of the measurement field. The light source is configured to provide a substantially spherical wavefront or a substantial planar wavefront, at least at the measurement field. Preferably, the apparatus with the light source that provides a substantial planar wavefront, comprises a gimbal mount. Furthermore, the invention relates to a method for measuring a perfusion in a tissue using the handheld laser-based perfusion imaging apparatus as described above.

1-16. (canceled)

17: A handheld laser-based perfusion imaging apparatus comprising a light source and an imaging device which are arranged in a fixed orientation to each other in said apparatus, wherein
the light source is configured for projecting a beam of coherent light onto a measurement field at a predetermined distance spaced apart from the apparatus, and
the imaging device is configured for recording intensity maps of the measurement field,
wherein the light source comprises one or more optical components arranged for configuring the beam of coherent light so as to comprise a spherical wavefront, wherein the wavefront is convex towards the measurement field.

18: The handheld laser-based perfusion imaging apparatus according to claim 17, wherein the one or more optical components are configured to position a center of the spherical wavefront at or near a pivot point of said handheld laser-based perfusion imaging apparatus and/or a pivot point of a user of said handheld laser-based perfusion imaging apparatus.

19: The handheld laser-based perfusion imaging apparatus according to claim 17, wherein the one or more optical components comprises a single mode optical fibre, which is configured for emitting a diverging light beam.

20: The handheld laser-based perfusion imaging apparatus according to claim 17, wherein the one or more optical components comprises a focusing lens and a pinhole aperture arranged at a position where the focusing lens focusses the beam of coherent light.

21: The handheld laser-based perfusion imaging apparatus according to claim 17, wherein the light source is configured to provide a substantially collimated light beam and wherein the one or more optical components comprises a lens, preferably a negative lens, for converting the substantially collimated light beam in a diverging light beam with a spherical wavefront.

22: The handheld laser-based perfusion imaging apparatus according to claim 17, wherein the light source comprises one or more further optical components which are configured for converting the spherical wavefront into a planar wavefront.

23: The apparatus according to claim 22, wherein the one or more further optical components comprises collimating lenses and/or a beam expander.

24: The apparatus according to claim 22, wherein the apparatus comprises a gimbal mount with a handle, wherein at least the light source and the imaging device are pivotally isolated from the handle.

25: A handheld Laser Speckle Contrast Imaging (LSCI) apparatus or Laser Doppler Perfusion Imaging (LDPI) apparatus comprising a light source and an imaging device which are arranged in a fixed orientation to each other in said apparatus, wherein
the light source is configured for projecting a beam of coherent light onto a measurement field at a predetermined distance spaced apart from the apparatus,
the imaging device is configured for recording the speckle intensity maps of the measurement field,
wherein the light source is configured to provide a substantial planar wavefront, at least at the measurement field.

26: The apparatus according to claim 25, wherein the one or more further optical components comprises collimating lenses and/or a beam expander.

27: The apparatus according to claim 25, wherein the apparatus comprises a gimbal mount with a handle, wherein at least the light source and the imaging device are pivotally isolated from the handle.

28: The apparatus according to claim 17, wherein the apparatus comprises one or more targeting light sources which are configured to project one or more targeting light beams onto the measurement field, preferably wherein the one or more targeting light sources comprises two cross-line laser modules which are configured to illuminate the boundaries of the measurement field.

29: A method for measuring a perfusion in a tissue using a handheld Laser Speckle Contrast Imaging (LSCI) apparatus, wherein the method comprises the steps of:
projecting a beam of coherent light onto a measurement field at a predetermined distance spaced apart from the apparatus,
recording time integrated speckle intensity maps of the measurement field,
wherein the apparatus comprises a light source comprising one or more optical components for configuring the beam of coherent light to comprise a spherical wavefront at least at the measurement field, wherein the wavefront is convex towards the measurement field.

30: The method for measuring a perfusion in a tissue according to claim 29, wherein the light source comprises one or more further optical components which are configured for converting the spherical wavefront into a planar wavefront.

31: A method for measuring a perfusion in a tissue using a handheld Laser Doppler Perfusion Imaging (LDPI) apparatus, wherein the method comprises the steps of:
projecting a beam of coherent light onto a measurement field at a predetermined distance spaced apart from the apparatus,
recording a series of speckle images at a high frame rate and/or short exposure times in order to obtain a series of substantially non-blurred speckle images, wherein the apparatus comprises a light source comprising one or more optical components for configuring the beam of coherent light to comprise a spherical wavefront at least at the measurement field, wherein the wavefront is convex towards the measurement field.

32: The method for measuring a perfusion in a tissue according to claim 31, wherein the light source comprises one or more further optical components which are configured for converting the spherical wavefront into a planar wavefront.

33: The method for measuring a perfusion in a tissue according to claim 32, wherein the apparatus comprises a gimbal mount.

34: A method for measuring a perfusion in a tissue using a handheld Laser Speckle Contrast Imaging (LSCI) apparatus, wherein the method comprises the steps of:
projecting a beam of coherent light onto a measurement field at a predetermined distance spaced apart from the apparatus,
recording time integrated speckle intensity maps of the measurement field,
wherein the apparatus comprises a light source, wherein the light source is configured to provide a substantial planar wavefront, at least at the measurement field.

35: The method for measuring a perfusion in a tissue according to claim 34, wherein the apparatus comprises a gimbal mount.

36: A method for measuring a perfusion in a tissue using a handheld Laser Doppler Perfusion Imaging (LDPI) apparatus, wherein the method comprises the steps of:
projecting a beam of coherent light onto a measurement field at a predetermined distance spaced apart from the apparatus,
recording a series of speckle images at a high frame rate and/or short exposure times in order to obtain a series of substantially non-blurred speckle images, wherein the apparatus comprises a light source, wherein the light source is configured to provide a substantial planar wavefront, at least at the measurement field.

37: The method for measuring a perfusion in a tissue according to claim 36, wherein the apparatus comprises a gimbal mount.
